(19) United States
(12) Patent Application Publication
(10) Pub. No.: US 2024/0033463 A1
(43) Pub. Date: Feb. 1, 2024

(54) RECTAL ANAESTHESIA DELIVERY DEVICE AND METHOD

(71) Applicant: NEW MEDTEK DEVICES PTY LTD, Sydney (AU)

(72) Inventors: David Zachary LUBOWSKI, Edgecliff (AU); Joseph Ignatius Maria VAN DER PLAS, Victoria (AU)

(73) Assignee: NEW MEDTEK DEVICES PTY LTD, Sydney (AU)

(21) Appl. No.: 18/254,221

(22) PCT Filed: Nov. 3, 2021
(86) PCT No.: PCT/AU2021/051294

§ 371 Date: May 24, 2023

(30) Foreign Application Priority Data
Nov. 25, 2020 (AU) 2020904358

(51) Int. Cl. A61M 19/00; A61M 39/10; A61M 25/01; A61M 25/02; A61M 39/02; A61B 17/34; A61N 1/36

(52) U.S. Cl. CPC A61M 19/00; A61M 39/10; A61M 25/0194; A61M 25/02; A61M 39/0247; A61B 17/3415; A61N 1/36071; A61M 2039/0273; A61M 2039/0276; A61B 2017/3445; A61B 2017/347

(57) ABSTRACT

A nerve stimulating trocar assembly (500) for insertion into tissue of a patient is disclosed, the nerve stimulating trocar assembly including an elongate trocar body (510) and a nerve stimulator (550). The trocar body (510) extends from a proximal end (511) to a distal end (512) and has an elongate open channel (520), which extends along a length of the trocar body (510). The nerve stimulator (550) has a shaft (560) extending from a proximal end (561) to a distal end (562), and at least one electrode (570) at or adjacent to the distal end (562) of the shaft (560). The open channel (520) of the trocar body (510) is configured to receive both a catheter tube and the shaft (560) of the nerve stimulator (550) such that the catheter is releasably secured between the trocar body (510) and the nerve stimulator (550) in an assembled configuration. Also disclosed are trocar assemblies and a method for positioning a catheter in a target tissue site.

### TECHNICAL FIELD

The present invention relates to devices and methods for use in the management of patient pain through nerve anaesthesia. More specifically, the invention relates to an delivery device for an implantable catheter system for the delivery of anaesthetic to an area of tissue around a nerve of a patient following a haemorrhoidectomy or similar surgery.

### BACKGROUND

Haemorrhoids are enlarged, prolapsing anal cushions, which can result in bleeding, itching and pain and can affect more than 50% of people at some point in their lives.

In severe cases, treatment may involve surgery to physically remove the haemorrhoids, a procedure known as a haemorrhoidectomy. Under local or general anaesthesia, incisions are made in the tissue of a patient around the haemorrhoid. The vessels inside the haemorrhoid are tied off to prevent bleeding, and the haemorrhoid is removed.

Haemorrhoidectomy is generally considered a day procedure, with the patient typically released from hospital to return home within 24 hours. However, due to the extensive network of nerves within the anal canal, postoperative pain can be significant for the patient.

Currently, after haemorrhoidectomy surgery, local anaesthetic is injected into the area immediately after surgery with the effect lasting for up to 24 hours. The patient is then given oral opioids/narcotics to manage pain which may persist for many weeks. Complete recovery from the procedure can vary between patients, from between 2 weeks to 2 months.

Severe postoperative pain not only requires opioid use, which may have unwanted risks and side effects, but may also prolong the hospital stay and affect the comfort and wellbeing of the patient.

It is therefore desirable to provide a controlled delivery of a medicament, such as an anaesthetic, to a nerve branch of a patient following a haemorrhoidectomy, or similar (other) surgery of the anal canal.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

According to one aspect of the present disclosure, there is provided a nerve stimulating trocar assembly for insertion into tissue of a patient comprising: an elongate trocar body extending from a proximal end to a distal end, the trocar body having an elongate open channel which extends along a length of the trocar body; a nerve stimulator having a shaft extending from a proximal end to a distal end, and at least one electrode at or adjacent to the distal end of the shaft; wherein the open channel of the trocar body is configured to receive both a catheter tube and the shaft of the nerve stimulator such that the catheter is releasably secured between the trocar body and the nerve stimulator in an assembled configuration.

In some embodiments, the electrode may partially extend beyond the distal end of the trocar body. In such embodiments, the electrode may also act as a tissue separator during insertion of the trocar. For example, a distal tip of the electrode may have a curved or wedge-shaped profile. Further, a width, height or diameter of the electrode may be smaller than a width, height or diameter of the trocar body, such that the electrode encounters less initial resistance to passing through the tissue. The electrode may have a substantially rounded or curved distal tip. The electrode tip may be devoid of sharp or bevelled edges, for example. The non-sharp shape of the distal end of electrode tip and the distal end of the trocar body may allow the electrode and trocar of the nerve stimulation assembly to pass through tissue without causing damage to anatomical structures, such as the pudendal nerves. This may be in contrast to conventional insertion of infusion catheters using a sharp trocar which could cut or otherwise damage anatomical structures, such as nerves, during insertion.

The channel may have a substantially U-shaped cross section.

The assembly may further comprise a locking mechanism for releasably locking the nerve stimulator to the trocar body in the assembled configuration. The locking mechanism may be provided at or adjacent to the distal end of the trocar body.

In further embodiments, the locking mechanism comprises a notched region in a distal wall of the trocar body and a transversely extending locking bar of the electrode. The transversely extending locking bar of the nerve stimulator may be received in the notched region.

When in an assembled configuration, the locking bar of the nerve stimulator is typically held in a tight engagement with the distal wall. The notched region of the distal wall may include a stop surface to prevent the locking bar advancing towards the distal end of the trocar body when in the assembled configuration. The tight engagement of the locking bar with the distal wall and the stop surface together prevent any substantial longitudinal movement of the nerve stimulator shaft relative to the trocar body in the assembled configuration.

The tight engagement of the locking bar with the distal wall may be released by pulling the nerve stimulator shaft in a direction away from the distal end of the trocar body, wherein the force applied to the nerve stimulator shaft is sufficient to cause the locking bar to release its engagement with the distal wall.

The notched region of the distal wall may include a ramped surface proximal to the stop surface. When a force is applied to the nerve stimulator shaft in a direction away from the distal end of the trocar body, the locking bar typically releases from its tight engagement and rides up the ramped surface such that the nerve stimulator may be withdrawn entirely from the trocar body.

In the assembled configuration, the nerve stimulator overlies the catheter and substantially locks the catheter within the channel of the trocar body. A distal end of the catheter may be positioned set back proximally from the distal end of the trocar body.

The assembly may further comprise a handle at a proximal region of the nerve stimulating trocar assembly. Typically, the assembly is made of two separate components including a base and a slider. The base may be connected to the proximal end of the trocar body. In one embodiment, the base is integral with the trocar body to form a single unit. The base may include an elongate channel extending longitudinally along its length. The channel of the base is typically aligned with the open channel of the trocar body. Further, the channel of the base of the handle is typically in fluid communication with the open channel such that together they may both receive a length of a catheter therein.

The base and the slider may be slidably connected to each other in the assembled configuration. Where the base in integral with the trocar body and therefore fixed relatively to the trocar body, the slider may slide longitudinally relative to the base. In the assembled configuration, the slider and the base may be in relatively locking engagement to each other to prevent sliding of the slider. However, the engagement between the two parts may be released to allow the side to move relative to the base.

The slider may be comprised of a pair of shells, which may mate to form a housed configuration. The slider may be connected to the proximal end of the nerve stimulator shaft. The slider may be configured to receive the nerve stimulator shaft in the housed configuration. One or more shells of the slider may include a notched region, configured to engage a locking bar of the nerve stimulator shaft, thereby to inhibit relative longitudinal movement between the nerve stimulator shaft and the slider when the nerve stimulator shaft is received in the slider in the housed configuration. In this embodiment, sliding of the slider relative to the base in a direction away from the distal end of the trocar body will apply a force on the nerve stimulator shaft. A sufficient force will release the transverse locking bar of the electrode from its tight engagement with the distal wall of the trocar body and up the ramped surface to allow withdrawal the nerve stimulator from the trocar body. In use, this allows a surgeon to fully withdraw the nerve stimulator once the trocar body is in a desired location within the tissue of a patient.

The slider may include a housing having a passage extending from a proximal end opening to a distal end opening. A proximal length of the nerve stimulator shaft may be received through the distal end opening. The proximal end opening of the slider may receive an electrical lead for electrical connection with the proximal end of the nerve stimulator shaft. The electrical lead may be connected to an energy source to deliver energy to the electrode of the nerve stimulator.

According to another aspect of the present disclosure, there is provided a trocar assembly having: an elongate trocar shaft extending from a proximal end to a distal, tissue separating end and including an adapter at the proximal end to connect the trocar shaft to a catheter; a handle configured to house a length of the trocar shaft adjacent to its proximal end in a housed configuration and to disengage from the trocar shaft in a release configuration; wherein in the housed configuration, the handle includes an access port for connection of the adapter with the catheter.

In some embodiments, the trocar assembly may further comprise a catheter connector configured to connect to the adapter. In some embodiments, the catheter connector comprises a male connecting portion. The adapter typically comprises a receiving portion to receive the male connecting portion. The catheter connector may also include a female connecting portion at an opposite end to the male connecting portion. The female connecting portion may be configured to receive an end of the catheter.

In some embodiment, the catheter connector may include two male connecting portions and two female connecting portions. In such embodiments, the adapter may include two receiving portions to receive the two male connecting portions of the connector.

In other embodiments, a proximal portion of the trocar shaft may include an adapter configured to receive the ends of one or more catheter tubes. For example, the proximal portion of the trocar shaft may define one or more bores, apertures or slits configured to receive the ends of the one or more catheter tubes. The proximal portion of the trocar shaft may be configured to be crimped, thereby to secure the one or more catheter tubes within the adapter.

For example, in some embodiments, the proximal portion of the trocar shaft may include one or more walls defining a central bore, or lumen. The bore or lumen may be configured to receive the ends of one or more catheter tubes. The walls of the proximal region of the trocar shaft may be configured to be crimped, compressing the catheter ends within the bore thereby to secure the catheter tubes to the trocar shaft.

In other embodiments, a proximal region of the trocar shaft may define one or more apertures configured to receive the end of one or more catheter tubes therethrough (in the manner of threading a needle, for example). In some embodiments, the proximal region of the trocar shaft may define a transverse aperture having a widened portion and a narrowed portion. The trocar shaft may be configured to be crimped at the proximal end, adjacent the aperture, thereby to secure the ends of one or more catheter tubes therein.

In alternative embodiments, the trocar shaft may include two or more tines at a proximal end of the shaft. The tines may define a slit therebetween, adapted for receiving one or more catheter tubes. One or more of the tines may be adapted to be crimped (by bending the tines towards each other, for example) to secure the ends of the one or more catheter tubes in the slit. One or more of the tines may include one or more notches for more securely gripping the one or more catheter tubes.

In some embodiments, one or more surfaces of the trocar shaft may be roughened for more securely gripping the one or more catheter tubes.

The handle may be configured to house a length of the trocar shaft adjacent to its proximal end in the housed configuration and to disengage from the trocar shaft in the release configuration. In the housed configuration, the handle may include an access port for connection of the adapter with a catheters.

The handle of the assembly may be configured to facilitate easier insertion of the trocar shaft through the tissue of the patient. Once the distal tip of the trocar shaft has passed through an exit incision, the handle may be removed, by causing it to move to its release configuration. This allows the trocar shaft and attached catheter tube to be drawn through the tissue of the patient.

The handle may comprise two components, mateable with each another to form the housed configuration. In some embodiments, the handle is comprised of a pair of shells. The handle may comprise a snap-fit connection system for fastening the shells together in the housed configuration. In some embodiments, one of the shells comprises one or more projections receivable in corresponding recesses in the other shell portion to prevent relative movement between the shell portions in the housed configuration.

The handle may further comprise a release mechanism to release the one or more projections from their corresponding recesses. The release mechanism may be in the form of a button which may be depressed to release the shells from their housed engagement.

The handle further comprises interior ribs configured to engage corresponding notches on a proximal portion of the trocar shaft to prevent relative axial movement of the handle and the trocar shaft when in the housed configuration.

In some embodiments, the handle may be configured to crimp a portion of the trocar shaft. For example, the handle may comprise one or more interior projections or surfaces configured to impinge on and deform a region of the trocar shaft as the shells are mated to crimp the region of the shaft.

According to another aspect of the present disclosure, there is provided a method for positioning a catheter in a target tissue site to deliver a medicament to a patient after haemorrhoid surgery, the method including:
providing a first catheter which extends from a proximal end to a distal end, the first catheter having a sidewall which defines an internal lumen, the distal end of the catheter having one or more apertures for the release of the medicament to the target tissue site;
making a first incision in the skin on one side of the anus;
making a lateral incision in the skin of the thigh of the patient;
connecting the proximal end of the first catheter to an end of a first trocar; tunnelling the first trocar from the first incision through the tissue and through the lateral incision;
pulling the first trocar from the lateral incision until a desired length of the first catheter is pulled through the lateral incision, such that the proximal end of the first catheter extends from the lateral incision and the distal end of the first catheter extends from the first incision;
disconnecting the proximal end of the first catheter from the trocar;
connecting the distal end of the first catheter to a stimulator trocar, the stimulator trocar comprising an elongate body and a nerve stimulating electrode configured to stimulate nerves in the target tissue site;
advancing the stimulator trocar, together with the distal end of the first catheter, through the tissue of the patient;
actuating the nerve stimulator at a determined frequency;
adjusting the positioning of the stimulator trocar and the distal end of the first catheter until a physical contraction of the external anal sphincter is observed at a frequency that correlates with the determined frequency of the nerve stimulator;
identifying the location of the nerve stimulator and the distal end of the catheter where the physical contraction of the external anal sphincter is achieved as the first target tissue site;
disconnecting the stimulator trocar from the first catheter and withdrawing the stimulator trocar through the first incision leaving the distal end of the catheter implanted in the first target tissue site;
connecting the proximal end of the catheter to a reservoir of medicament and infusing the medicament through the internal lumen of the catheter to deliver the medicament to the first target tissue site.

The first trocar typically includes a removable handle which is removed from the trocar after the step of tunnelling the first trocar from the first incision through the tissue and through the lateral incision.

According to another aspect of the present disclosure, there is provided a method for positioning first and second catheters in respective first and second target tissue sites to deliver a medicament to a patient after haemorrhoid surgery, the method including:
providing a first catheter which extends from a proximal end to a distal end, the first catheter having a sidewall which defines an internal lumen, the distal end of the first catheter having one or more apertures for the release of the medicament to the first target tissue site;
providing a second catheter which extends from a proximal end to a distal end, the second catheter having a sidewall which defines an internal lumen, the distal end of the second catheter having one or more apertures for the release of the medicament to the second target tissue site;
making a first incision in the skin on one side of the anus;
making a second incision in the skin on an opposite side of the anus to the first incision;
connecting the proximal end of the first catheter to a first trocar;
tunnelling the first trocar from the first incision through the tissue across the midline of the patient and through the second incision;
pulling the first trocar from the second incision until a desired length of the first catheter is pulled through the second incision, such that the proximal end of the first catheter extends from the second incision and the distal end of the first catheter extends from the first incision;
disconnecting the proximal end of the first catheter from the first trocar; connecting the proximal end of the first catheter to a second trocar;
connecting the proximal end of the second catheter to the second trocar; making a lateral incision in the skin of the thigh of the patient, the lateral incision being on the same side of the anus as the second incision;
tunnelling the second trocar from the second incision through the tissue and through the lateral incision;
pulling the second trocar from the lateral incision until a desired length of the first and second catheters is pulled through the lateral incision, such that the proximal ends of the first and second catheters extend from the lateral incision, the distal end of the first catheter extends from the first incision and the distal end of the second catheter extends from the second incision,
connecting the distal end of the first catheter to a stimulator trocar, the stimulator trocar comprising an elongate body and a nerve stimulator configured to stimulate nerves in the target tissue site;
advancing the stimulator trocar and the distal end of the first catheter through the tissue of the patient;
actuating the nerve stimulator at a determined frequency;
adjusting the positioning of the stimulator trocar and the distal end of the first catheter until a physical contraction of the external anal sphincter is observed at a frequency that correlates with the determined frequency;
identifying the location of the nerve stimulator and the distal end of the first catheter where physical contraction of the external anal sphincter is achieved as the first target tissue site;
disconnecting the first catheter from the stimulator trocar and withdrawing the stimulator trocar through the first incision leaving the distal end of the first catheter implanted in the first target tissue site;
connecting the distal end of the second catheter to the stimulator trocar; advancing the stimulator trocar and the distal end of the second catheter through the second incision through tissue of the patient;
actuating the nerve stimulator at the determined frequency;
adjusting the positioning of the stimulator trocar and the distal end of the second catheter until a physical contraction of the external anal sphincter is observed at a frequency that correlates with the determined frequency;
identifying the location of the nerve stimulator and the distal end of the second catheter where physical contraction of the external anal sphincter is achieved as the second target tissue site;
disconnecting the second catheter from the stimulator trocar and withdrawing the stimulator trocar through the second incision leaving the distal end of the first catheter implanted in the second target tissue site;
connecting the proximal ends of the first and second catheters to a reservoir of medicament and infusing the medicament through the internal lumens of the first and second catheters to deliver the medicament to the first and second target tissue sites.

The first trocar again typically includes a removable handle which is removed from the trocar after the step of tunnelling the first trocar from the first incision through the tissue across the midline of the patient and through the second incision. The second trocar may also include a removable handle which is removed after the step of tunnelling the second trocar from the second incision through the tissue and through the lateral incision.

In another embodiment, rather than a connecting both catheters to the second trocar, one catheter may be connected to the second trocar and the other to a third trocar, each of which may pull the respective catheter through the lateral incision.

The target tissue site may comprise tissue adjacent to the anal and/or rectal branches of the pudendal nerves although any site adjacent to nerve of interest is envisaged. The first target tissue site may comprise tissue around the left pudendal nerve and the second target tissue site may comprise tissue around the right pudendal nerve or vice versa.

The medicament to be delivered may be an analgesic or anaesthetic agent. The analgesic or anaesthetic agent may include but is not limited to bupivacaine, lidocaine, ropivacaine, opioid analgesics such as buprenorphine, hydromorphone, ketobemidone, levomethadyl, levorphanol, mepiridine, methadone, morphine, nalbuphine, opium, oxycodone, pentazocine, phenoperi-dine, butorphanol, dextromoramide, dezocine, dextropropoxyphene, diamorphine, fentanyl, alfentanil, sufentanil, hydrocodone, piritramide, dextropropoxyphene, remifentanil, sufentanil, tilidine, tramadol, codeine, dihydrocodeine, meptazinol, dezocine, eptazocine, flupirtine or a combination thereof.

Typically the analgesic is ropivacaine. The ropivacaine may be delivered in solution with a concentration of 0.25%, or 0.5% or 0.75% or 1.0%. The ropivacaine may be in a concentration of between 0.25 to 0.5% or 0.5% to 1.0%. In some embodiments, the concentration of ropivacaine may exceed 0.75%.

The analgesic may be delivered at a dose of 20 mls per day (24 hours). Alternatively, the analgesic may be delivered at a dose of 1 ml or 5 ml or 10 mls or 15 mls or 25 mls or 30 mls per day. The dose may, in some embodiments exceed 30 mls per day including 40 mls, 50 mls, 60 mls, 70 mls, 80 mls, 90 mls or 100 mls per day. In some embodiments, the dose may exceed 100 mls per day.

The analgesic may be delivered to the patient continuously over the time period. Alternatively, it may be delivered in dosage intervals within the time period. The dosage intervals may occur every 1, 5, 10, 20, 30, 40 or 50 minutes. Further, the dosage intervals may occur hourly, every 2 hours, every 3 hours, every 4 hours, every 5 hours, every 6 hours, every 7 hours, every 8 hours, every 9 hours, every 10 hours, every 11 hours or every 12 hours.

The dosage intervals may range of from 30 secs up to 6 hours. The dosage intervals may be 30 minutes, 1 hour, 2 hours, 3 hours, 4 hours, 5 hours of 6 hours. In some cases, the dosage intervals may be longer than 6 hours. For example the dosage interval may be 7 hours, 8 hours, 9 hours, 10 hours, 11 hours or 12 hours. Further, the dosage intervals may exceed 12 hours and may be from 12 hours to 23 hours.

The time period may range from 1 day to 3 months. For example, the time period may be 1 week, 2 weeks, 3 weeks, 4 weeks, 5 weeks, 6 weeks, 7 weeks, 2 months or 3 months.

The analgesic may be delivered in an initial burst followed by continuous delivery thereafter or alternatively followed by dosage intervals.

The initial burst may enable a larger initial dose to be delivered over an initial period of time to achieve an immediate pain relief for the patient. The initial burst may deliver a percentage of the daily dose. For example, the initial burst may deliver 10%, 20%, 30%, 40%, 50%. 60%, 70%, 80% or 90% of the daily dose. The regimen may be such that such an initial burst occurs daily, or every second, third, fourth, fifth or sixth day during the time period. Further, an initial burst may occur once every week, every 2 weeks, every three weeks.

Alternatively, an initial burst may only occur once, for example, on the first day of treatment.

In a preferred embodiment, the catheters are relatively flexible. However, the catheters may have sufficient rigidity so as to permit passage through the body to the target tissue site.

The catheters may comprise a single aperture at or adjacent the distal end. Alternatively the catheters may include a plurality of apertures at or adjacent the distal end. The plurality of apertures may be positioned around the circumference of the catheters.

The apertures may be evenly spaced relative to each other or unevenly spaced. Further, the apertures may be arranged in ring-like arrangements around the circumference of the catheters. The apertures may extend a length of the catheter from the distal end. Alternatively, the apertures may be positioned along one side of a catheter. In such an embodiment, the catheter may be oriented during use such that the apertures are adjacent and facing the nerve or nerve branch.

The apertures of a catheter may be arranged in a number of configurations in addition to the ring like arrangement described above. For example, the apertures may be arranged in a helical or partially helical arrangement along a length of the catheter.

An aperture may be formed in a distal end of the catheter. In one embodiment, the internal lumen may be open ended to allow fluid to flow therethrough and out of the distal end through the lumen.

In a further aspect, there is provided a surgical kit comprising:
first and second catheters configured for implantation in respective first and second target tissue sites of a patient, each catheter extending from a proximal end to a distal end and each having a sidewall which defines an internal lumen, said distal ends having one or more apertures;
a first tunnelling trocar configured for attachment to the distal end of the first catheter;
a second tunnelling trocar configured for simultaneous attachment to the distal ends of both the first and second catheters;
a stimulator trocar comprising an elongate body and a nerve stimulator configured to stimulate nerves in a target tissue site.

The surgical kit may further comprise a sheet for application to the patient's skin around the surgical site, the sheet having an adhesive surface for adhering to the skin of the patient and a series of frangible regions for removal of parts of the sheet.

The surgical kit may further comprise a retainer for securing one or more catheters at an exit wound site of a patient, the retainer comprising a retainer body having a lower, skin facing surface and a guide surface configured to receive one or more catheters, the retainer further comprising a plurality of clips to secure a length of the one or more catheters to the retainer.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

By way of example only, embodiments are now described with reference to the accompanying drawings, in which:

FIG. 1a shows a perspective view of a wearable apparatus for delivery of a medicament device according to an embodiment of the present disclosure;

FIG. 1b shows a perspective view of a catheter tube of the wearable apparatus of FIG. 1a;

FIGS. 2a and 2b show front and perspective views, respectively, of a distal end of the catheter tube of FIG. 1b;

FIGS. 2c and 2d show front and perspective views, respectively, of a distal end of another catheter tube of the wearable apparatus of FIG. 1a;

FIG. 3a shows a side view of a trocar assembly according to an embodiment of the present disclosure;

FIG. 3b shows a perspective view of a distal end of the trocar assembly of FIG. 3a;

FIG. 3c shows a perspective view of a proximal end of the trocar assembly of FIG. 3a, a catheter connector and the distal end of a catheter tube of the wearable apparatus of FIG. 1;

FIG. 4a shows a side view of a trocar assembly according to another embodiment of the present disclosure;

FIG. 4b shows a perspective view of a distal end of the trocar assembly of FIG. 4a;

FIG. 4c shows a perspective view of a proximal end of the trocar assembly of FIG. 4a, a catheter connector and the distal ends of two catheter tubes of the wearable apparatus of FIG. 1;

FIG. 5a shows a perspective view of the catheter assembly of FIG. 4a;

FIG. 5b shows a cross-sectional view of the handle of the trocar assembly of FIG. 4a;

FIG. 6a shows a partial perspective exploded view of the trocar assembly of FIG. 4a;

FIG. 6b shows a partial perspective view of the trocar assembly of FIG. 4a;

FIG. 7 shows a side view of a nerve stimulating trocar assembly according to an embodiment of the present disclosure;

FIGS. 8a and 8b show perspective views of the nerve stimulating trocar assembly of FIG. 7 in a disassembled state and an assembled state, respectively;

FIGS. 8c and 8d show a distal end of the nerve stimulating trocar assembly of FIG. 7 in a disassembled state and an assembled state, respectively;

FIGS. 9a and 9b show a side view and a cross-sectional view, respectively, of the nerve stimulating trocar assembly of FIG. 7;

FIG. 9c shows the nerve stimulating trocar assembly of FIG. 7 in use;

FIGS. 10a-h show steps in one embodiment of a method of implanting two catheter tubes in a patient;

FIGS. 11a-c are perspective views of a retainer of the wearable apparatus of FIG. 1;

FIG. 12 shows a surgical kit according to an embodiment of the present disclosure;

FIGS. 13a and 13b show front and side views, respectively, of a trocar shaft according to an embodiment of the present disclosure;

FIGS. 13c and 13d show front and side views, respectively, of a distal end of the trocar shaft of FIG. 13a;

FIGS. 13e and 13f show cross-sectional side and end views, respectively, of a proximal end of the trocar shaft of FIG. 13a;

FIGS. 14a and 14b show front and side views, respectively, of a trocar shaft according to an embodiment of the present disclosure;

FIGS. 14c and 14d show front and side views, respectively, of a distal end of the trocar shaft of FIG. 14a;

FIGS. 14e and 14f show cross-sectional side and end views, respectively, of a proximal end of the trocar shaft of FIG. 14a;

FIG. 15 shows a perspective view of the proximal end of the trocar shaft of FIG. 14a engaging a catheter tube;

FIGS. 16 and 17 show the proximal end of the trocar shaft of FIG. 14a received in a handle;

FIGS. 18a-e show side and perspective views of a proximal end of a trocar shaft according to another embodiment of the present disclosure;

FIGS. 19a-d show side and perspective views of a proximal end of a trocar shaft according to another embodiment of the present disclosure;

FIGS. 20a and 20b show perspective views of a top shell and base shell, respectively, of a trocar handle according to an embodiment of the present disclosure;

FIGS. 21a and 21b show perspective views of a top shell and base shell, respectively, of a trocar handle according to another embodiment of the present disclosure; and

FIGS. 22a and 22b show perspective views of a top shell and base shell, respectively, of a trocar handle according to another embodiment of the present disclosure.

### DESCRIPTION OF EMBODIMENTS

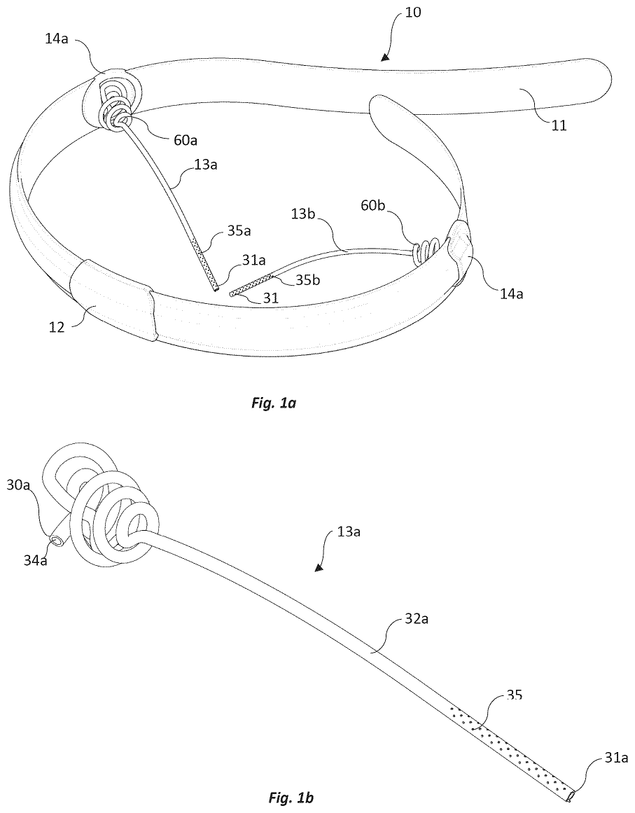

A wearable apparatus according to one embodiment of the present disclosure is illustrated as 10 in FIG. 1a. The wearable apparatus 10 includes a belt 11, pump 12, catheters 13a, 13b and housing units 14a, 14b. As discussed in more detail below, the belt 11 is attachable to a patient around their waist and catheters 13a, 13b are configured to be implanted in the patient with distal ends 31a and 31b at or adjacent to a right and left pudendal nerve respectively to deliver pain relief medicament directly to the nerves.

In other embodiments, as discussed in more detail below, each of the catheters may exit the body on the same side and connect to a single housing unit.

In the depicted embodiments, both proximal ends 30a, 30b of catheters 13a, 13b, respectively, are in fluid connection with pump 12 such that the pump 12 pumps a medicament into both catheters 13a, 13b. The proximal end of the two catheters 13a and 13b may be connected either directly or indirectly to pump 12. The medicament may be pumped in unison, delivering medicament to both catheters 13a and 13b at substantially the same time. Alternatively, the pump may alternate between delivery of medicament to catheter 13a and catheter 13b at different time periods.

Pump 12 may be positioned either at the front of the belt such that it sits adjacent to the navel region of a wearer or at the rear of belt 11 to sit adjacent the small of the back of a wearer, or at the side of the belt. Pump 12 may draw medicament from a single reservoir or it may draw from multiple reservoirs.

FIG. 1b depicts a view of one of the catheters 13a. The same features equally apply to catheter 13b and catheter 13a has been selected purely for illustrative purposes. Catheter 13a extends from a proximal end 30a to a distal end 31a and has a sidewall 32a which defines an internal lumen 34a. Adjacent distal end 31a, the catheter sidewall has a plurality of apertures 35a for the delivery of a medicament. Apertures 35a provide a fluid flow path from the internal lumen 34a to the outside of catheter 13a.

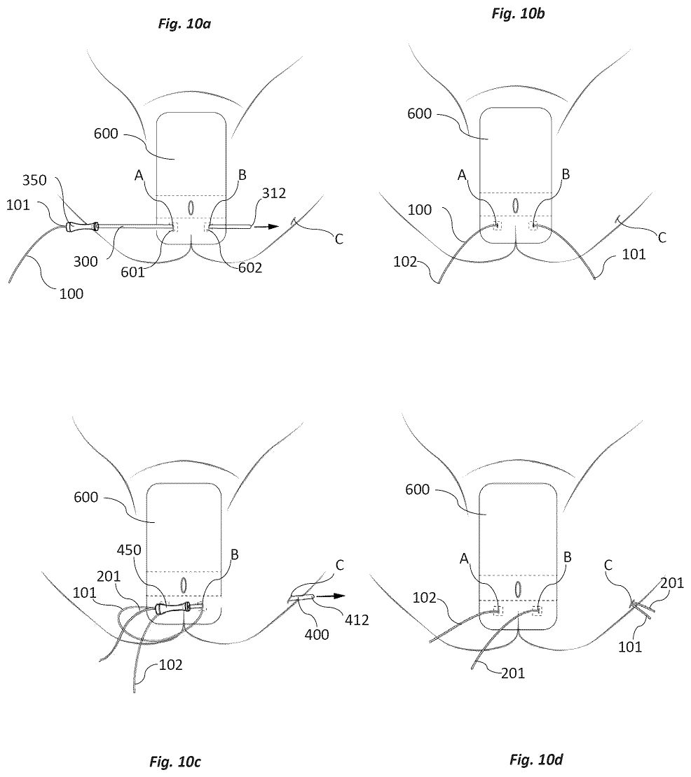

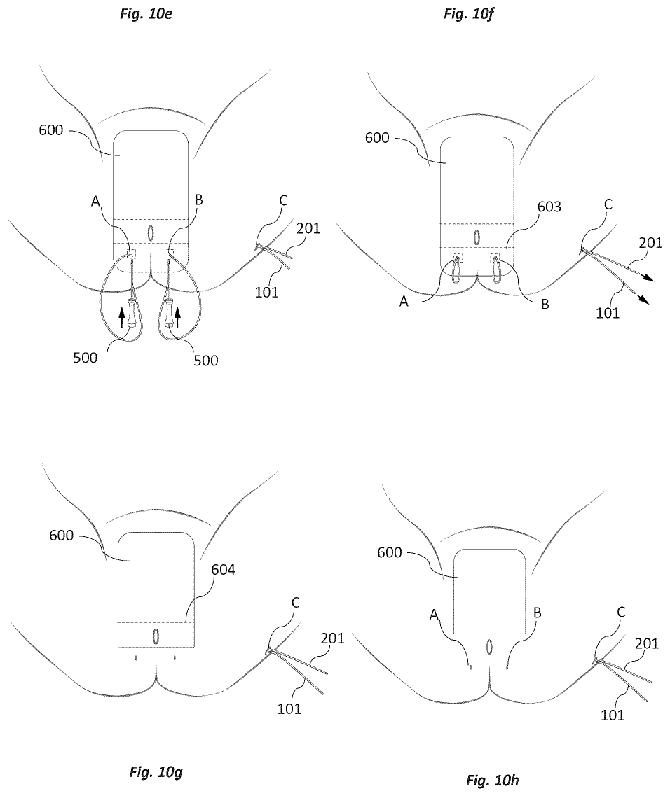

In the embodiment shown in FIG. 1b, a coiled region 60a of catheter 13a extends from proximal end 30a. Coiled region 60a may be configured such that it is positioned beneath the skin of the patient when the catheter 13a is implanted. The coiled structure acts as a strain relief and prevents pulling or tugging at the wound site which could open the wound and cause discomfort for the patient and an increased risk of infection. Typically, the coiled region 60a extends a length of the catheter 13a such that it sits in the fatty layer beneath the skin of the patient. Coiled regions 60a and 60b may include a shape memory material such that when inserted in the body, said regions adopt the coiled configuration. In other embodiments, the catheter may be substantially straight. In other embodiments a strain relief (e.g. a coiled portion) in the catheter may be extracorporeal. One such embodiment is described in further detail below with reference to FIGS. 10a to 10c.

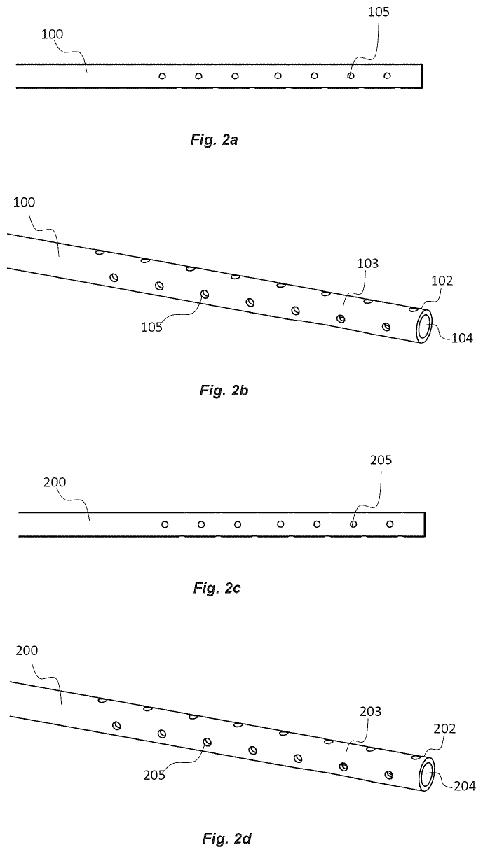

FIGS. 2a and 2b show part of a catheter 100 according to an embodiment of the present disclosure. Catheter 100 extends from a proximal end 101 to a distal end 102 and has a sidewall 103 which defines an internal lumen 104. One or more apertures 105 are formed in the sidewall adjacent distal end 102 to allow for fluid in the internal lumen to pass into the surrounding tissue.

FIGS. 2c and 2d show part of a catheter 200 according to an embodiment of the present disclosure. Catheter 200 extends from a proximal end 201 to a distal end 202 and has a sidewall 203 which defines an internal lumen 204. One or more apertures 205 are formed in the sidewall adjacent distal end 202 to allow for fluid in the internal lumen to pass out and into the surrounding tissue.

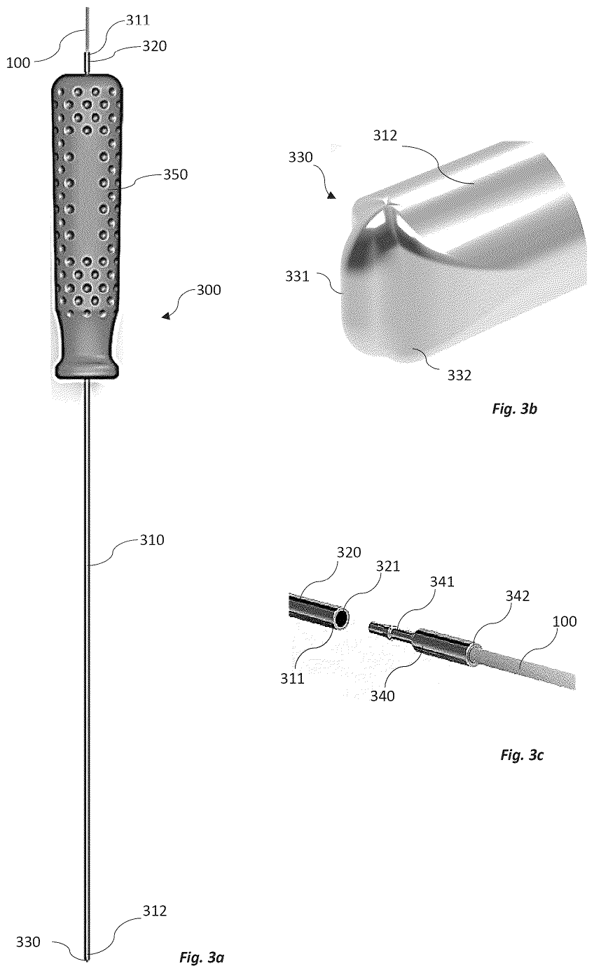

FIG. 3a shows a trocar assembly 300 according to an embodiment of the present disclosure. The trocar assembly 300 has an elongate trocar shaft 310 extending from a proximal end 311 to a distal end 312.

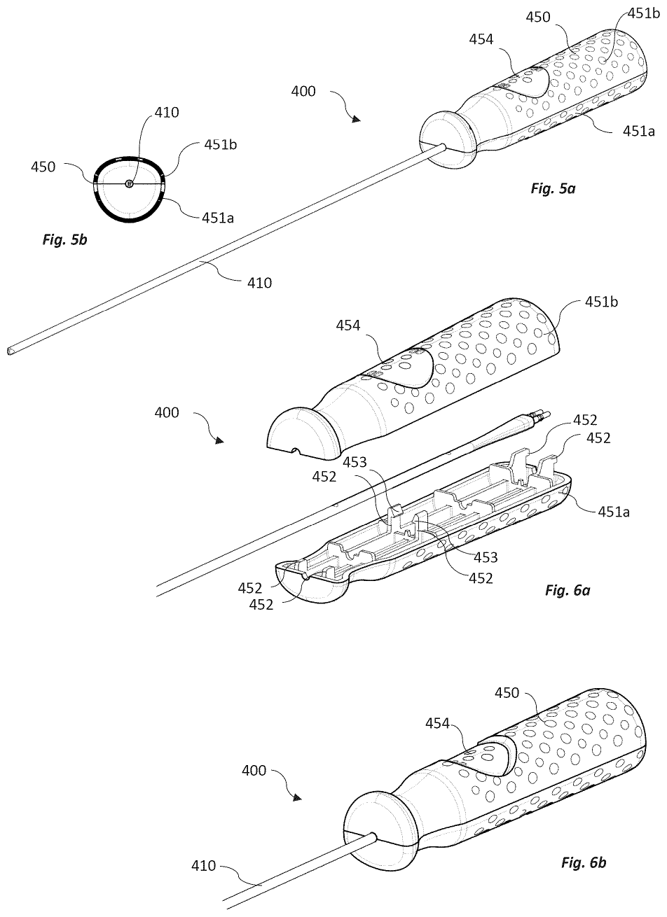

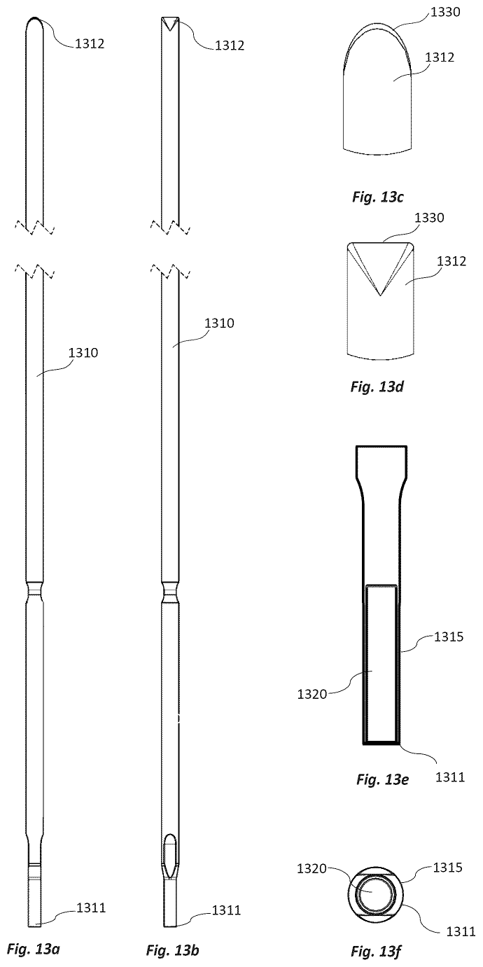

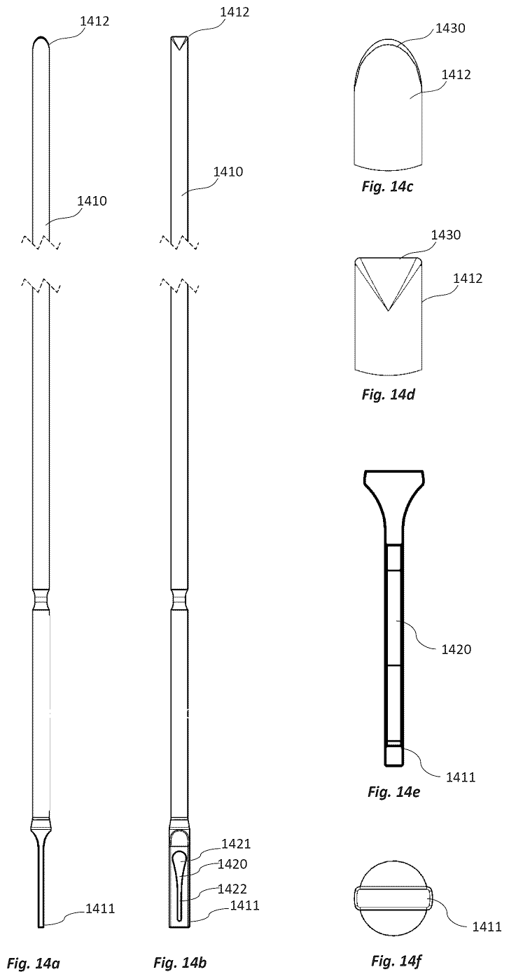

The distal end 312 of the trocar assembly 300 includes a tissue separator 330. An enlarged view of tissue separator 330 is shown in FIG. 3b. In the illustrated embodiment, the tissue separator 330 comprises a substantially wedge-shaped portion of the distal end 312 of the trocar. The tissue separator 330 is curved and devoid of sharp or cutting edges which may damage tissue during insertion. As shown in FIG. 5, the tissue separator 330 comprises a stepped tip. The stepped tip comprises at least a distal step 331 and a proximal step 332. The distal step 331 is substantially wedge shaped and has a generally sharper curvature than the proximal step 332 which is substantially blunt. Alternately, the tip of the trocar may be a more rounded structure devoid of the steps 331 and 332, for example, as shown in FIGS. 13a-d and FIG. 14a-d.

An adapter 320 is provided at the proximal end for connecting the trocar shaft 310 to a catheter. In some embodiments, trocar assembly 300 further comprises a catheter connector 340 configured to connect to the adapter 320. One example of a catheter connector 340 is shown in FIG. 3c. In the illustrated embodiment, the catheter connector 340 comprises a male connecting portion 341. The adapter 320 typically comprises a corresponding receiving portion 321 to receive the male connecting portion 341. The catheter connector 340 also includes a female connecting portion 342 at an opposite end to the male connecting portion 341. In this embodiment, the female connecting portion 342 is configured to receive an end of a catheter 100.

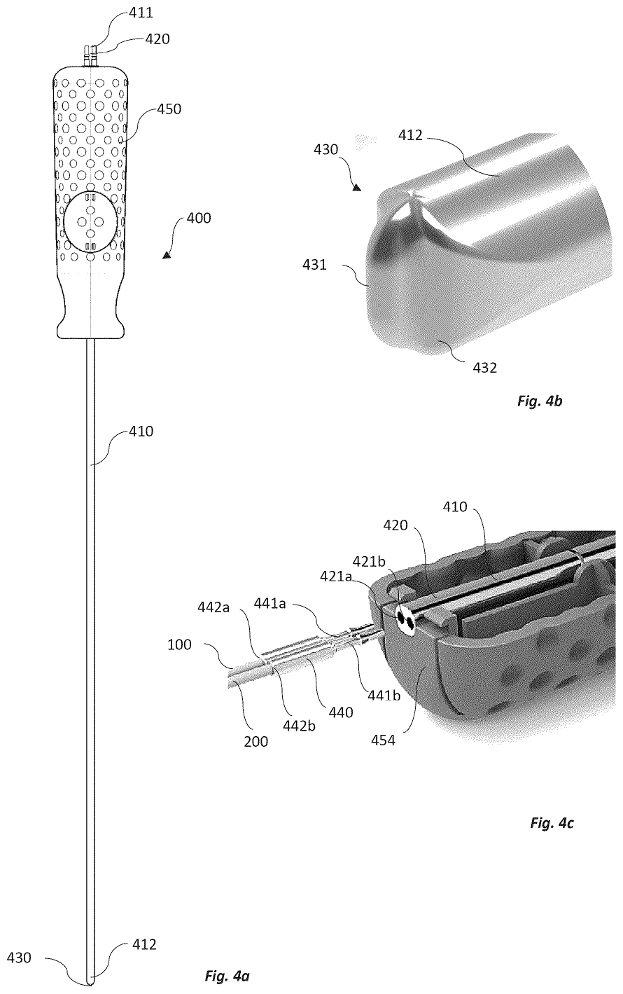

FIG. 4a shows another embodiment of a trocar assembly 400. The trocar assembly 400 has an elongate trocar shaft 410 extending from a proximal end 411 to a distal end 412. The shaft 410 of the trocar assembly 400 may be thicker than the shaft 310 of the trocar assembly 300.

The distal end 412 of the trocar assembly 400 includes a tissue separator 430, as shown in FIG. 4b. The features of the tissue separator 430 may be substantially as for tissue separator 330, discussed above.

An adapter 420 is provided at the proximal end 411 for connecting the trocar shaft 410 to one or more catheters. In some embodiments, trocar assembly 400 further comprises a catheter connector 440 configured to connect to the adapter 420. One example of a catheter connector 440 is shown in FIG. 4c. In the illustrated embodiment, the catheter connector 440 comprises two male connecting portions 441a, 441b. In this embodiment, the adapter 420 includes two receiving portions 421a, 421b to receive the two male connecting portions 441a, 441b of the connector 440. In other embodiments, a single male connecting portion 441 may be used. In other embodiments, the connection may be reversed. That is, the adapter 420 may include male connecting portions (for example, as shown in FIG. 4a) adapted to be received in a corresponding receiving portion of the catheter connector 440.

The catheter connector 440 shown in FIG. 4c also includes two female connecting portions 442a, 442b at an opposite end to the male connecting portions 441a, 441b. In this embodiment, the female connecting portions 442a, 442b are each configured to receive an end of one of the catheters 100, 200 such that two catheters may be connected to the catheter connector 440.

Each of the trocar assemblies 300, 400 further includes a handle 350, 450. FIG. 5a shows the trocar assembly 400 including the handle 450, while FIG. 5b shows a cross section of the handle 450. FIGS. 6a and 6b show the handle 450 in various configurations. The features of handle 450 discussed below may apply equally to handle 350 of trocar assembly 300.

In the illustrated embodiment, the handle 450 is comprised of a pair of shells 451a, 451b which mate together via a snap-fit connection system to form a housed configuration. Shell 451a comprises projections 452a, 452b which are receivable in corresponding recesses of shell 451b to align the shell members and fasten the shells to each other and to prevent relative movement between the shell portions in the housed configuration. Projections 452b comprise resilient arm members having shoulder portions 453 which latch in corresponding recesses in shell 451b to fasten the shells 451a, 451b to each other in housed engagement.

The handle further comprises a release mechanism 454 to release the shoulder portions 453 of the resilient arm members 452 from their corresponding recesses, thereby to release the two shells from their housed engagement. In the illustrated embodiment, the release mechanism 454 is a button, which may be depressed to release the shells 451a, 451b. When depressed, the button pushes against sloped surfaced of the resilient arm members 452, causing them to deform out of latching engagement with the recesses, releasing the shells 451a, 451b from their engagement. In the embodiment of FIGS. 6a-6d, the button is positioned on a top of the shell 451b. In other embodiments, a button may be positioned elsewhere. For example, FIG. 4c shows a button positioned on a rear portion of shell 451a.

In the housed configuration, the handle 450 houses a length of the trocar shaft 410 adjacent to its proximal end 411 (e.g. as shown in FIG. 6b). In the released configuration, the handle 450 disengages from the trocar shaft 410 (e.g. as shown in FIG. 6c). In the housed configuration, the handle 450 includes an access port for connection of the adapter 420 with one or more catheters (e.g. via the catheter connector 440).

The handle 450 of the assembly may be configured to facilitate easier insertion of the trocar shaft 410 through the tissue of the patient. Once the distal tip 412 of the trocar shaft 410 has passed through an exit incision, the handle 450 may be removed, by causing it to move to its release configuration. This allows the trocar shaft 410 and attached catheter tube (or tubes) to be drawn through the tissue of the patient.

The handle 450 further comprises interior ribs 455 configured to engage corresponding notches on a proximal portion of the trocar shaft 410 to prevent relative axial movement of the handle 450 and the trocar shaft 410 when in the housed configuration.

FIGS. 13a-f show another embodiment of a trocar shaft 1310 according to the present disclosure. The trocar shaft 1310 is elongate and extends from a proximal end 1311 to a distal end 1312. The distal end 1312 of the trocar shaft 1310 includes a tissue separator 1330. An enlarged view of tissue separator 1330 is shown in FIGS. 13c and 13d. In the illustrated embodiment, the tissue separator 1330 comprises a substantially wedge-shaped portion of the distal end 1312 of the trocar shaft 1310. The tissue separator 1330 has a rounded front profile, as shown in FIG. 13c, and a blunt side profile, as shown in FIG. 13d. This combination of round and flattened profiles may enable the tissue separators 1330 to effectively tunnel through tissue by parting the tissues, that is, by pushing anatomical features to the side rather than cutting or damaging the tissues as a sharper trocar tip might do.

An adapter in the form of a bore 1320 is provided for connecting the trocar shaft 1310 to a catheter. The bore 1320 is defined by a peripheral wall 1315 at the proximal end 1311 of the trocar shaft 1310 and is configured to receive one or more catheter tubes therein. The wall 1315 is configured to be crimped, thereby to secure the one more catheter tubes within the bore 1320.

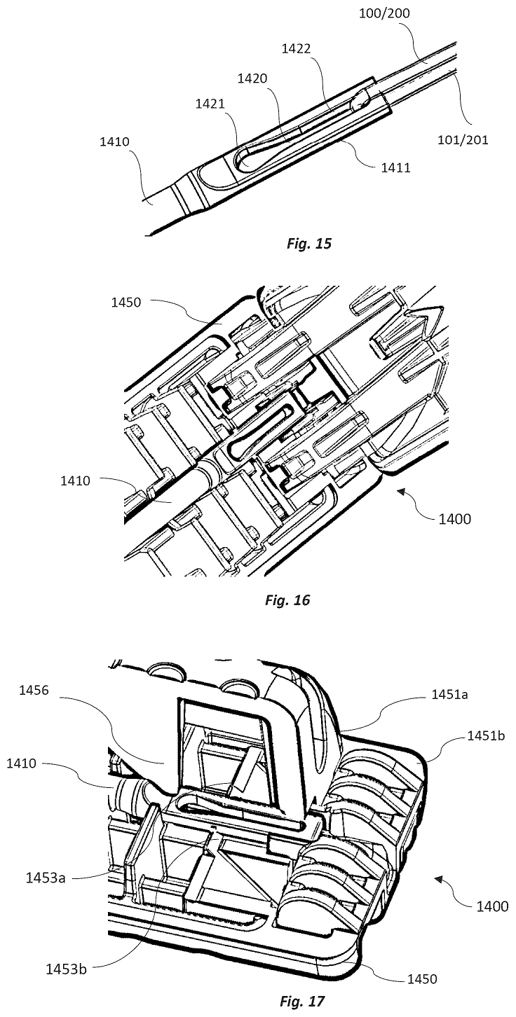

FIGS. 14a-f and FIG. 15 show another embodiment of a trocar shaft 1410 according to the present disclosure. The trocar shaft 1410 may be comprised in a trocar assembly 1400 including a handle 1450 as shown in FIGS. 16 and 17. Referring to FIGS. 14a and 14b, the trocar shaft 1410 is elongate and extends from a proximal end 1411 to a distal end 1412. The distal end 1412 of the trocar shaft 1410 includes a tissue separator 1430, as shown in FIGS. 14c and 14d, which is substantially the same as tissue separator 1330 described above. In this embodiment, the proximal end 1411 of the trocar shaft 1410 defines a catheter adapter including an aperture 1420 for engaging one or more catheters. Aperture 1420 extends through a flattened portion of the proximal end 1411, transverse to a longitudinal axis of the trocar shaft 1410. Aperture 1420 includes an enlarged portion 1421 and a narrowed portion 1422. To secure a catheter tube, such as catheter tube 100, to the trocar shaft 1410, a proximal end 101/201 of the catheter tube may be advanced through the enlarged portion 1421 in the manner of threading a needle. The catheter tube 100 may be subsequently pulled into the narrowed portion 1422, as shown in FIG. 15. The proximal end 1411 of the trocar shaft 1410 may be crimped to secure the catheter tube 100 in the aperture 1420.

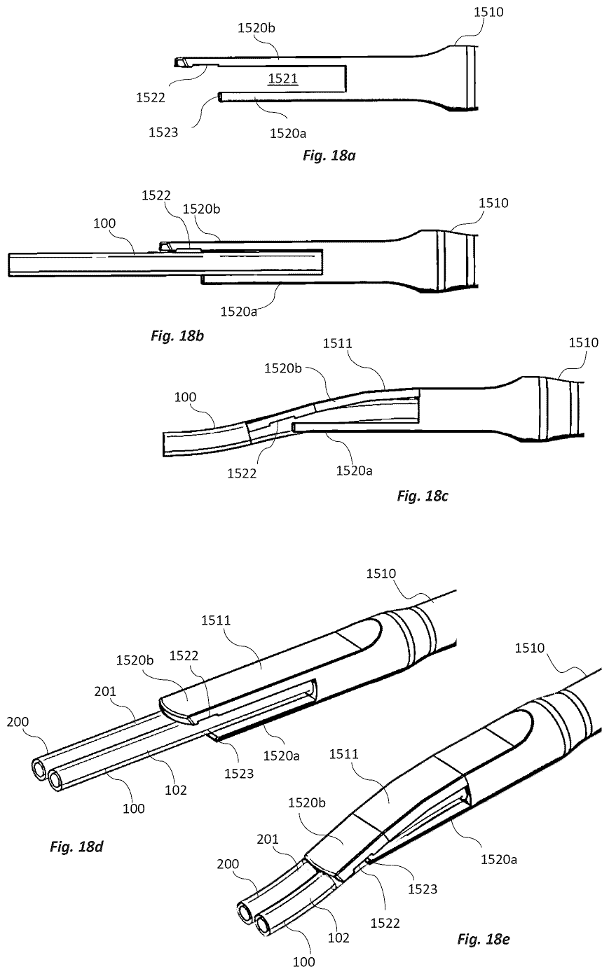

An alternative embodiment of a trocar shaft 1510 is shown in FIGS. 18a-e. In this embodiment, the proximal end 1511 of the trocar shaft 1510 includes a catheter adapter comprising a pair of tines 1520a and 1520b, defining a slit 1521 there between. The upper tine 1520b extends proximally beyond the lower tine 1520a. The slit 1521 is configured to receive the ends of one or more catheter tubes therein. In the illustrated example, the slit receives the ends 102, 201 of two tubes 100, 200, as shown in FIG. 18d. However, the slit may also receive a single tube. Once the ends 102, 201 of the catheter tubes 100, 200 are received within the slit 1521, the proximal end 1511 of the trocar shaft 1510 may be crimped to secure the tubes 100, 200 to the shaft 1510 by bending the upper tine 1520b towards the lower tine 1520a, as shown in FIG. 18e. In other embodiments, the lower tine may be bent towards the upper tine, or both tines may be bent towards each other. Upper tine 1520b defines a notch 1522, adapted for gripping the catheter tubes 100, 200. The notch 1522 is positioned such that, when the tines 1520a, 1520b are crimped, the notch 1522 aligns with a proximal end 1523 of lower tine 1520a. As such, when the proximal end 1511 of the trocar shaft 1510 is crimped, the proximal end 1523 of the lower tine 1520a compresses the ends 102, 201 of the catheter tubes 100, 200 into the notch 1522, thereby to more securely attach the tubes 100, 200 to the trocar shaft 1510.

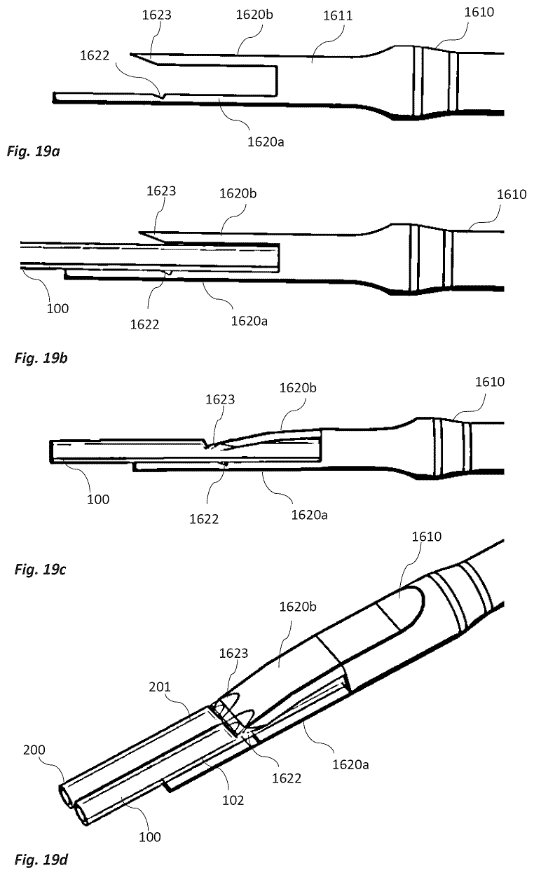

Another embodiment of a trocar shaft 1610 is shown in FIGS. 19a-d. The trocar shaft 1610 is similar to trocar shaft 1510 described above, having a lower tine 1620a and an upper tine 1620b defining a catheter tube receiving slit 1621 therebetween. However, in this embodiment, the lower tine 1620a extends in a proximal direction beyond the upper tine 1620b. The lower tine 1620a defines a notch 1622. When the upper tine 1620b is bent toward the lower tine 1620a to secure catheter tubes 100, 200 therebewteen, a proximal end 1623 of the upper tine 1620b is aligned with the notch 1622. As such, when the proximal end 1611 of the trocar shaft 1610 is crimped, the proximal end 1623 of the upper tine 1620a compresses the ends 102, 201 of the catheter tubes 100, 200 into the notch 1622, thereby to more securely attach the tubes 100, 200 to the trocar shaft 1610.

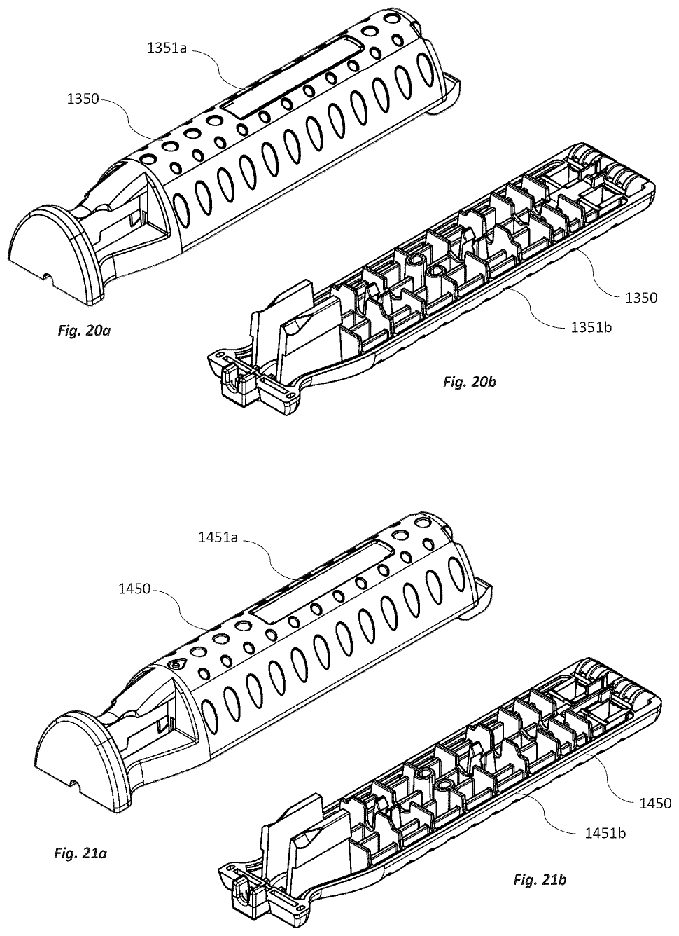

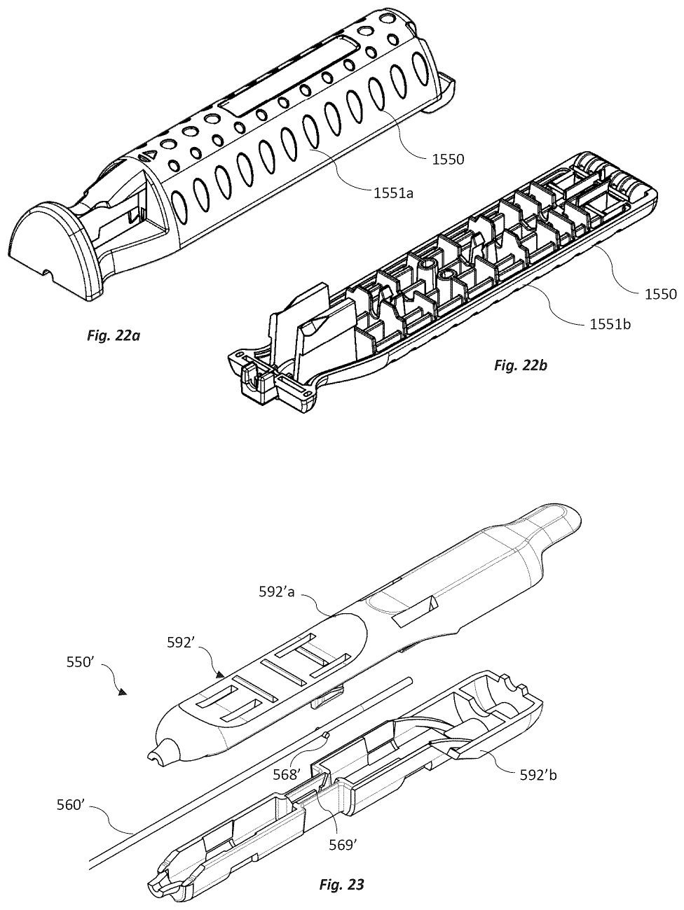

Each of the trocar shafts 1410, 1310, 1510 is receivable in a respective handle 1450, 1350, 1550 as shown in FIGS. 20a-22b. Handles 1350, 1450, 1550 are similar to handles 350 and 450 described above. In these embodiments, however, each handle 1350, 1450, 1550 is configured to crimp the proximal ends 1311, 1411, 1511 of the trocar shafts 1310, 1410, 1510 to secure one or more catheter tubes thereto. In other embodiments, the proximal ends 1311, 1411, 1511 of the trocar shafts 1310, 1410, 1510 may be manually crimped.

Handle 1350 is comprised of a pair of shells 1351a, 1351b (as shown in FIGS. 20a and 20b, respectively), which mate together to form a housed configuration. When the trocar shaft 1310 is received between the shells 1351a, 1351b with a catheter tube received in the bore 1320, projections within the top shell 1351a and the base shell 1351b impinge on the distal end 1311 of the trocar shaft as the shells mate, deforming wall 1315 and crimping the bore 1320 to secure the catheter tube therein.

Handle 1450 is similarly comprised of paired top shell 1451a and base shell 1451b, as shown in FIGS. 21a and 21b, respectively. When the trocar shaft 1410 is received between the shells, as shown in FIGS. 16 and 17, a pair of ramped surfaces 1453a and 1453b in the base shell 1451b flank the distal end 1411 of the trocar shaft 1410 adjacent the aperture 1420. The top shell 1451a includes a projection 1456 configured to impinge on the distal end 1411 of the trocar shaft 1410 as the shells 1451a and 1451b are mated, so as to push the trocar shaft 1410 into the base shell 1451b. As the trocar shaft 1410 is pushed into the base shell 1451b, the ramped surfaces deform the distal end 1411 of the trocar shaft, crimping the distal end 1411 (reducing the size of the aperture 1420) and securing the catheter tube 100 therein.

Similarly, handle 1550 is comprised of paired top and base shells 1551a, 1551b. (as shown in FIGS. 20a and 20b, respectively), which mate together to form a housed configuration. When the trocar shaft 1510 is received between the shells 1551a, 1551b with catheter tubes received in the slit 1521, projections within the top shell 1551a and the base shell 1551b impinge on and deform upper tine 1520b of the trocar shaft 1510 as the shells 1551a, 1551b mate, crimping the tines 1520a, 1520b together to secure the catheter tubes therein.

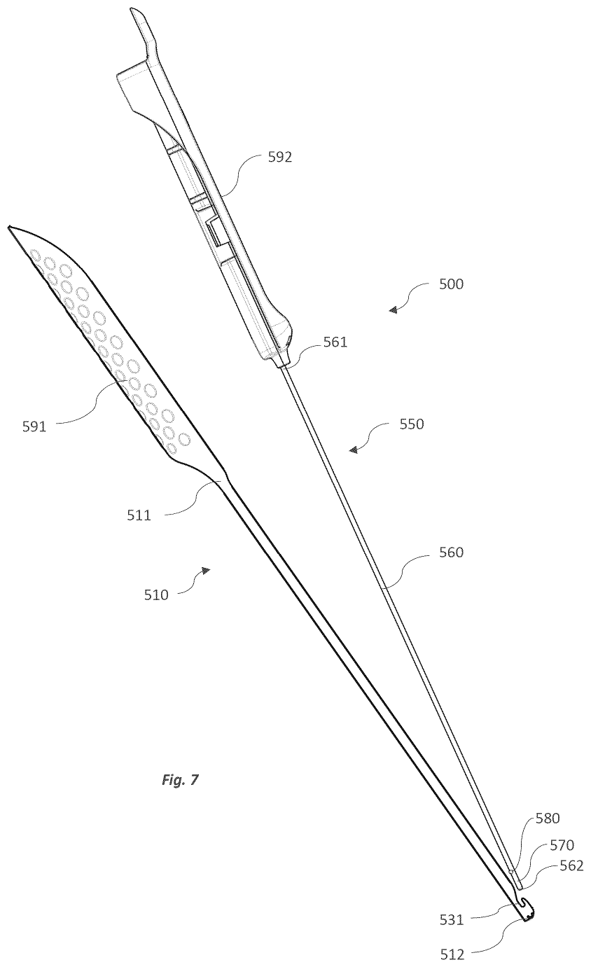

FIG. 7 shows a nerve stimulating trocar assembly 500 according to an embodiment of the invention in a disassembled configuration. The nerve stimulating trocar assembly 500 comprises an elongate trocar body 510 and a nerve stimulator 550.

The nerve stimulator 550 has a shaft 560 extending from a proximal end 561 to a distal end 562, and at least one electrode 570 at or adjacent to the distal end 562 of the shaft 560.

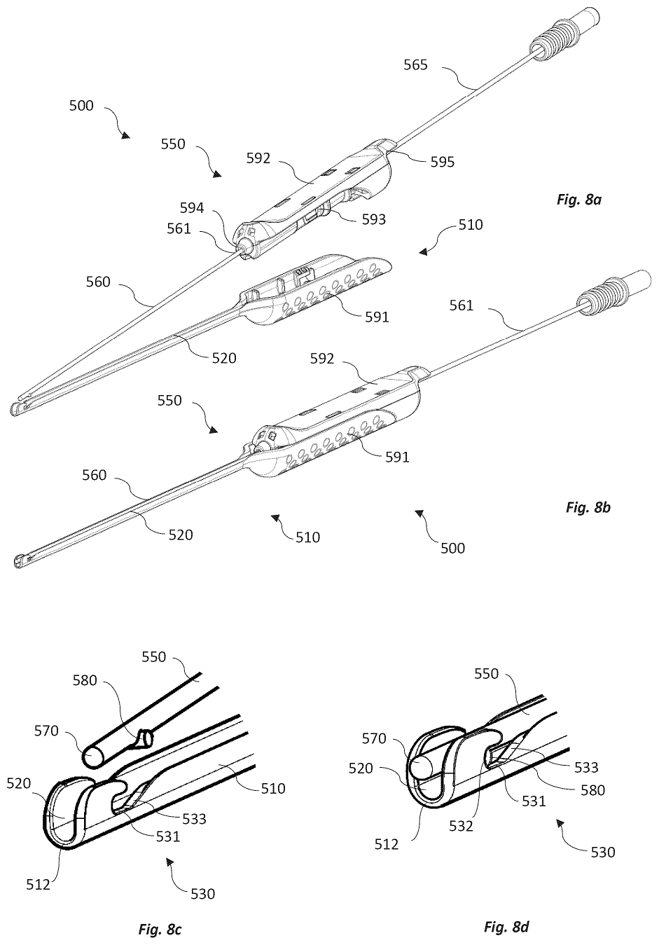

The elongate trocar body 510 extends from a proximal end 511 to a distal end 512. As shown in FIGS. 8a and 8b, the trocar body 510 has a U-shaped elongate open channel 520 which extends along a length of the trocar body 510. The open channel 520 of the trocar body 510 is shaped and configured to receive both a catheter tube and the shaft 560 of the nerve stimulator 550 such that the catheter is releasably secured between the trocar body 510 and the nerve stimulator 550 in an assembled configuration. When assembled with a catheter, the nerve stimulator 550 overlies the catheter and substantially locks the catheter within the channel 520 of the trocar body 510. A distal end of the catheter may be positioned set back proximally from the distal end 512 of the trocar body 510.

As shown in FIG. 8d, in the assembled configuration, the electrode 570 of the nerve stimulator 550 extends slightly beyond the distal end 512 of the trocar body 510. In this embodiment, the electrode 570 has a curved distal tip which acts as a tissue separator during insertion of the trocar body 510 into tissue of the patient. As the diameter of the of the electrode 570 is smaller than the diameter of the trocar body 510, the electrode encounters less initial resistance to passing through the tissue and inhibits damage of anatomical structures, such as nerves.

The nerve stimulating trocar assembly 500 further comprises a locking mechanism 530 adjacent the distal end 512 of the trocar body 510 for releasably locking the nerve stimulator 550 to the trocar body 510 in the assembled configuration. The locking mechanism 530 is shown in detail in FIGS. 8c and 8d. The locking mechanism 530 comprises a notched region 531 in a distal wall 513 of the trocar body 510 and a transversely extending locking bar 580 of the nerve stimulator 550.

When the trocar body 510 and nerve stimulator 550 are in an assembled configuration (for example, as shown in FIGS. 8b and 8d), the transversely extending locking bar 580 of the nerve stimulator 550 is received in the notched region 531. The locking bar 580 of the nerve stimulator 550 is typically held in a tight engagement with the distal wall 513 of the trocar body 510. The notched region 531 of the distal wall 513 may include a stop surface 532 to prevent the locking bar 580 advancing towards the distal end 512 of the trocar body 510 when in the assembled configuration. The tight engagement of the locking bar 580 with the distal wall 513 and the stop surface 532 together prevent any substantial longitudinal movement of the nerve stimulator shaft 560 relative to the trocar body 510 in the assembled configuration.

The tight engagement of the locking bar 580 with the distal wall 513 may be released by pulling the nerve stimulator shaft 560 in a direction away from the distal end 512 of the trocar body 510. The notched region 531 of the distal wall 513 includes a ramped surface 533 proximal to the stop surface 532. When sufficient force is applied to the nerve stimulator shaft 560 in a direction away from the distal end 512 of the trocar body 510, the locking bar 580 typically releases from its tight engagement with the distal wall 513 and rides up the ramped surface 533 such that the nerve stimulator 550 may be withdrawn entirely from the trocar body 510.

The assembly may further comprise a handle 590 at a proximal region of the nerve stimulating trocar assembly. Typically, the assembly is made of two separate components including a base 591 and a slider 592. The base 591 is connected to the proximal end 511 of the trocar body 510. In the illustrated embodiment, the base 591 is integral with the trocar body 510 to form a single unit. The base 591 includes an elongate channel extending longitudinally along its length. The channel of the base 591 is typically aligned with the open channel 520 of the trocar body. Further, the channel of the base of the handle is typically in fluid communication with the open channel such that together they may both receive a length of a catheter therein.

The base 591 and the slider 592 are longitudinally slidably connected to each other in the assembled configuration. In the assembled configuration, the slider 592 and the base 591 may be in relatively locking engagement to each other to prevent sliding of the slider 592. However, the engagement between the two parts may be released to allow the slider 592 to move relative to the base 591.

An alternative embodiment of a nerve stimulator 550′ including a shaft 560′ and a slider 592′ is shown in FIG. 23. The slider 592′ is comprised of two shells 592′a and 592′b which mate together to form a housed configuration. The nerve stimulator 550′ is substantially the same as nerve stimulator 550, except that, in this embodiment, the shaft 560′ includes a proximal locking bar 568′ (which may be in addition to a locking bat at a distal end, such as bar 580). The proximal locking bar 568′ is receivable in a corresponding notched region 569′ of shell 592′b. The notched region 569′ engages the proximal locking bar 568′ to inhibit longitudinal movement of the proximal locking bar 568′ relative to the slider 592′ when the shaft 560′ is received in the slider 592′ in the housed configuration.

As shown in FIGS. 8a and 8b, the slider 592 is connected to the proximal end 561 of the nerve stimulator shaft 560. In this embodiment, sliding of the slider 592 relative to the base 591 in a direction away from the distal end of the trocar body 510 will apply a force on the nerve stimulator shaft 560. A sufficient force will release the transverse locking bar 580 of the electrode 570 from its tight engagement with the distal wall 513 of the trocar body 510 and up the ramped surface 533 to allow withdrawal of the nerve stimulator 550 from the trocar body 510. In use, this allows a surgeon to fully withdraw the nerve stimulator 550 once the trocar body 510 is in a desired location within the tissue of a patient.

The slider 592 includes a housing 593 having a passage extending from a proximal end opening 594 to a distal end opening 595. A proximal length 561 of the nerve stimulator shaft may be received through the distal end 594 opening. The proximal end opening of the slider may receive an electrical lead 565 for electrical connection with the proximal end 561 of the nerve stimulator shaft. The electrical lead 565 may be connected to an energy source to deliver energy to the electrode 570 of the nerve stimulator 550.

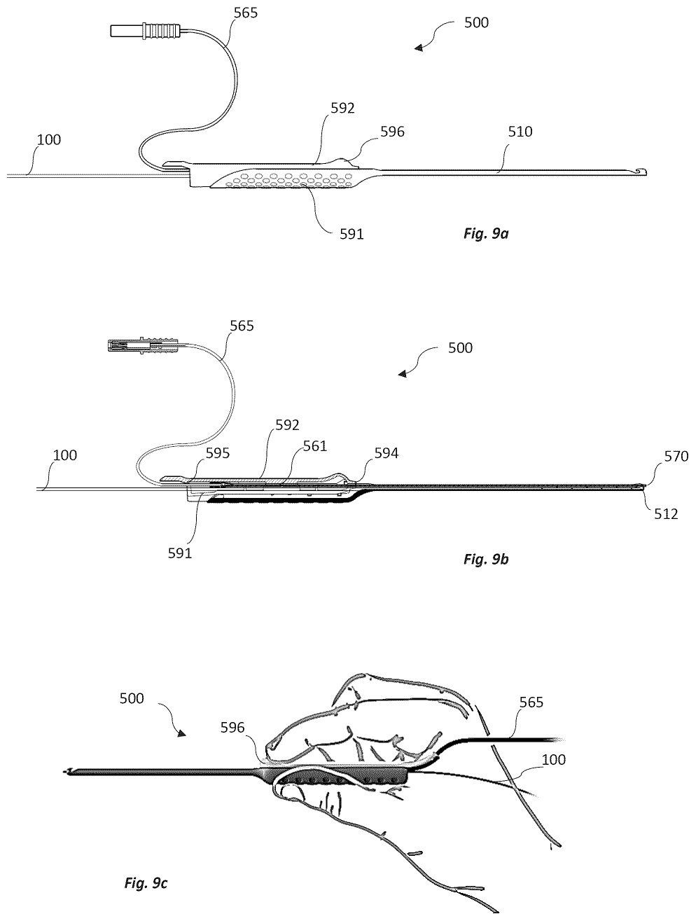

FIGS. 9a and 9b show the assembled nerve stimulating trocar assembly including a catheter 100.

In some embodiments, the slider 592 is provided with a distal grip portion 596 engageable by a user's finger to enable force to be exerted on the assembly in a distal direction, e.g., during insertion of the nerve stimulating trocar assembly 500 through tissue of a patient. FIG. 9c shows the assembly 500 as it might be gripped by a user's hand during insertion into a patient's tissue.

Turning to the method shown in FIGS. 10a-10h, the drawings depict a patient in position for an operation to surgically remove their haemorrhoids. The patient is typically placed on the operating table under general anaesthetic with muscle relaxation in the depicted modified lithotomy position. The perineum, pelvic area and thighs are prepared in a sterile field using a solution of iodine and alcohol.

An iodine-impregnated adhesive mat 600 is placed vertically from the pubis to cover the vagina/scrotum and the anal region. This area is kept sealed until catheters 100 and 200 are implanted in the desired position. The adhesive mat 600 comprises first and second frangible regions 601, 602 with serrations in the mat allowing a small area of the mat (here shown as a square but could be any shape) to be torn off, for access to the skin beneath. The surgeon makes a stab incision A at the first frangible region and then removes the second frangible region 602 of mat 600 and makes a second stab incision B at this region, on the other side of the anus to stab incision B. A lateral stab incision C is made at the lateral thigh.

A proximal end of catheter 100 is attached to trocar 300. Trocar 300 is tunnelled from incision A to incision B, through the patient's tissue across the midline of the patient as shown in FIG. 10a. Once the distal end of trocar 300 has exited the stab incision B, the surgeon may remove the handle 350 of trocar 300, and pull the trocar 300 by the distal end 312 to draw the trocar 300, together with catheter 100, through stab incision B until a desired length of catheter 100 extends from incision B. Catheter 100 is then disconnected from trocar 300. A length of catheter 100 adjacent proximal end 101 extends from incision point B and a length of catheter 100 adjacent distal end 102 extends from incision point A, as depicted in FIG. 10b.

The proximal end 201 of the catheter 200 is attached to trocar 400. Additionally, the proximal end 102 of catheter 100 is connected to trocar 400. Trocar 400 is then passed from incision B to incision C, as shown in FIG. 10c. Once the distal end of trocar 400 has exited the incision C, the surgeon may remove the handle 450 of trocar 400, and pull the trocar 400 by the distal end 412 to draw the trocar 400, together with catheters 100 and 200, through stab incision C until a desired length of catheters 100 and 200 extends from incision C. Catheters 100, 200 are then disconnected from trocar 400. At this point, a length of catheter 200 adjacent distal end 202 extends from incision point B, a length of catheter 100 adjacent distal end 102 extends from incision point A, and lengths of catheters 100, 200 adjacent proximal ends 101, 201 extend from incision point C, as depicted in FIG. 10d.

As shown in FIG. 10e, distal end 102 of catheter 100 is then assembled in nerve stimulating trocar assembly 500. The nerve stimulator 550 is locked into the trocar body 510 overlying catheter 100, securing distal end 102 under the locking bar 580. Catheter 100 and nerve stimulating trocar assembly 500 are then passed through incision A and angled to tunnel deeply into the tissue until at a region where the target nerve branches (e.g., the pudendal nerves) are located.

The energy source is then activated. In one embodiment, the energy source is an electrical energy source which delivers an electrical stimulus to electrode 570 of nerve stimulating trocar assembly 500. The electrical stimulus is typically between 3-5 mAmp and at a frequency of between 0.5-1.0 Hz. The surgeon watches for contraction of the external sphincter of the anus at a cycle rate corresponding to the applied electrical stimulus. The surgeon may move the trocar 500 and catheter 100 until this contraction is observed. This contraction indicates to the surgeon that the electrode 570 of nerve stimulating trocar assembly 500 is adjacent to the branches of the pudendal nerve and that the distal end 102 of the catheter 100 is thus in a position to infuse medicament from apertures 105 to the branches of the pudendal nerve.

The nerve stimulator 550 may then be separated from the nerve stimulating trocar assembly 500 by sliding the slider 592 of the handle 590 in a proximal direction. This applies tension on the nerve stimulator shaft 560, causing the locking bar 580 to disengage from the notched region 531 in the trocar body 510. The nerve stimulator 550 may then be withdrawn from the trocar body 510, and fully withdrawn from incision A. Withdrawal of the nerve stimulator 550 releases catheter 100 from the trocar body 510. Trocar body may then be withdrawn through incision A, careful not to dislodge distal end 102 from its position adjacent to the pudendal nerve branches.

This nerve stimulation process is then repeated with catheter 200 through incision B, as also shown in FIG. 10e. The process may be performed with the same nerve stimulating trocar assembly 500, or with a second nerve stimulating trocar assembly 500.

The proximal ends 101 and 201 of catheters 100 and 200 are then pulled in the direction shown by the arrows in FIG. 10f to pull the excess of each catheter at incisions A and B subcutaneously. The catheters are not pulled so far as to cause any dislodgement of the positioning of the distal ends 102 and 202 adjacent to the branches of the pudendal nerves.

The surgeon then removes a portion of the adhesive mat 600 by tearing along frangible line 503. The incisions A and B are then sealed.

The lengths of catheter that exit incision C may be retained by retainer 700, as shown in FIGS. 11a to 11c. Retainer 700 both protects the wound of the patient and prevents strain on catheters 100/200 which could cause them to be pulled out of the incision C.

Retainer 700 includes a retainer body 720 which comprises a partially circular structure having a relatively planar base surface 721 configured to sit on the skin (or on an adhesive film applied over incision C) and an opposed guide surface 722 to receive a length of the first catheter 100 and/or the second catheter 200. The guide surface 722 may be curved and terminate in an inner rim 723a and an outer rim 723b. A series of clips 730 retain the catheters 100 and/or 200 to the guide surface 722. The clips 730 may be formed integrally with the retainer body 720 or may be a separate structure attachable onto the retainer body 720. In the illustrated embodiments, the clips 730 include hinged portions 731 and a fastening mechanism comprising resiliently deformable projections 732 at one end of the clip 730 which latch in an aperture 733 at an opposite end of the clip 730. The hinged portions 731 may be made from a flexible material such that the clips are moveable between an open configuration in which the catheters may be laid along the guide surface 722 and a closed configuration in which clips 730 are folded around the catheters 100, 200 and the projections 732 latched in the apertures 733 to secure the catheters 100 and 200.

In the illustrated embodiment, the retainer 700 includes two clips 730a, 730b. The catheters may be positioned in a desired location in the retainer 700 before securing with the clips 730. FIGS. 11a-11c illustrated a sequence of steps to secure the catheters 100, 200 in the retainer 700. In FIG. 11a, both clips 730a, 730b are in the open configuration, with the catheters 100, 200 partially wound around the guide surface 722. In FIG. 11b, the catheters 100, 200 are in their final position and secured by one clip 730a which has been fastened in the closed configuration. In FIG. 11c the second clip 730b has been fastened in the closed configuration to secure the catheters 100, 200.

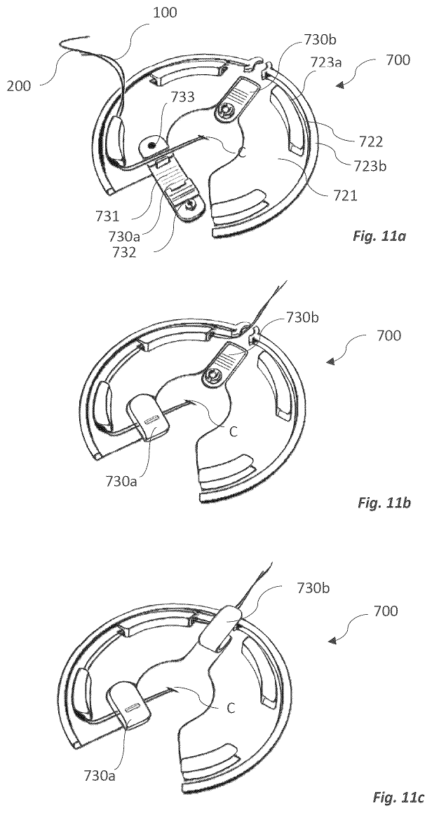

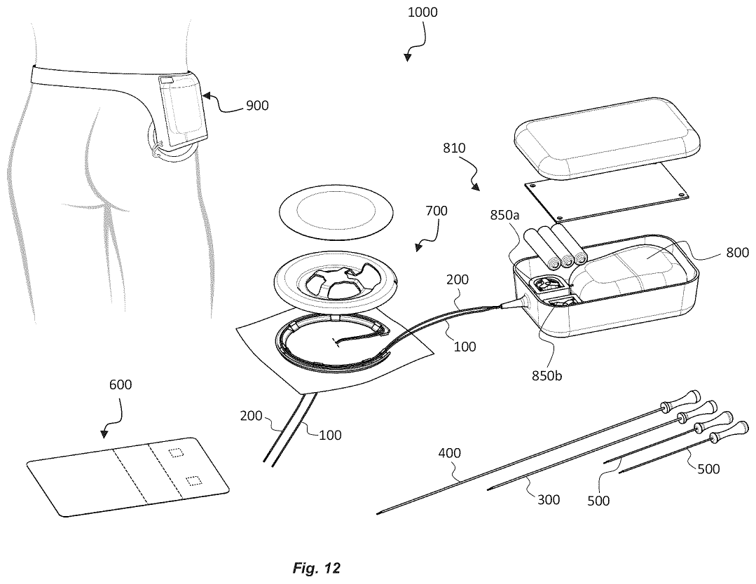

The proximal ends 101 and 201 of the catheters are ultimately connected to a medicament reservoir 800 which can be carried in a pouch 900 (e.g., as shown in FIG. 12) or like device anywhere on the patient's body. The patient may choose their preference for the location of pouch 900 prior to surgery and the retainer body 720 oriented accordingly such that the catheters 100 and/or 200 are directed towards the pouch 900.

FIG. 12 shows the parts of the system in a kit 1000 which includes the various parts of the system disclosed herein to allow the placement of catheters 100 and 200 adjacent to respective pudendal nerve branches to deliver medicament to a patient and alleviate pain after the surgical removal of haemorrhoids. Kit 1000 comprises two catheters 100 and 200, retainer 700, tunnelling trocar 300, tunnelling trocar 400, two nerve stimulating trocar assemblies 500, mat 600 and casing 810 housing medicament reservoir 800 and pumps 850a, 850b.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A nerve stimulating trocar assembly for insertion into tissue of a patient comprising:
an elongate trocar body extending from a proximal end to a distal end, the trocar body having an elongate open channel which extends along a length of the trocar body;
a nerve stimulator having a shaft extending from a proximal end to a distal end, and at least one electrode at or adjacent to the distal end of the shaft;
wherein the open channel of the trocar body is configured to receive both a catheter tube and the shaft of the nerve stimulator such that the catheter is releasably secured between the trocar body and the nerve stimulator in an assembled configuration.

2. The assembly of claim 1, wherein, in the assembled configuration, the electrode of the nerve stimulator is positioned at or adjacent to the distal end of the trocar body.

3. The assembly of claim 1, wherein the channel extends through the distal end of the elongate trocar body to define a distal end opening, wherein at least a portion of the electrode is exposed at the distal end opening of the trocar body.

4. The assembly of claim 2, wherein a distal tip of the electrode extends beyond the distal end of the trocar body.

5. The assembly of claim 1, wherein the channel is substantially U-shaped.

6. The assembly of claim 1, further comprising a locking mechanism for releasably locking the nerve stimulator to the trocar body in the assembled configuration.

7. The assembly of claim 6, wherein the locking mechanism is provided at or adjacent to the distal end of the trocar body.

8. The assembly of claim 6, wherein the locking mechanism includes a notched region in a distal wall of the trocar body, the notch configured to receive a transversely extending locking bar of the nerve stimulator.

9. The assembly of claim 8 wherein when in an assembled configuration, the locking bar of the nerve stimulator is in tight engagement with the distal wall.

10. The assembly of claim 9, wherein the notched region of the distal wall includes a stop surface to prevent the locking bar advancing towards the distal end of the trocar body.

11. The assembly of claim 8, wherein the locking mechanism further includes a release element to release the nerve stimulator from the trocar.

12. The assembly of claim 11 wherein the release element includes a ramped surface of the notched region of the distal wall along which the locking bar can slide in a direction away from the distal end of the trocar body.

13. The assembly of claim 6, wherein the locking mechanism secures a distal end of the catheter at or adjacent to the distal end of the trocar body.

14. The assembly of claim 1, further comprising a handle at a proximal region of the nerve stimulating trocar assembly.

15. The assembly of claim 14, wherein the handle includes a base and a slider.

16. The assembly of claim 15, wherein the base is connected to the proximal end of the trocar body and includes a channel in fluid communication with the open channel of the trocar body, said channel of the base configured to receive a length of the catheter therein.

17. The assembly of claim 15, wherein the base and the slider are slidably connected to each other in the assembled configuration.

18. The assembly of claim 17, wherein the base is fixed relative to the trocar body and the slider slides relative to the base.

19. The assembly of claim 15, wherein the slider is connected to the proximal end of the nerve stimulator shaft.

20. The assembly of claim 19, wherein the slider further includes a housing to receive an electrical lead connected to an energy source and wherein the proximal end of the nerve stimulator shaft is electrically connected to the electrical lead.

21. The assembly of claim 15, wherein sliding movement of the slider relative to the base causes the nerve stimulator shaft to move along the open channel of the trocar body.

22. A trocar assembly having:
an elongate trocar shaft extending from a proximal end to a distal, tissue separating end and including an adapter at the proximal end to connect the trocar shaft to a catheter;
a handle configured to house a length of the trocar shaft adjacent to its proximal end in a housed configuration and to disengage from the trocar shaft in a release configuration;
wherein in the housed configuration, the handle includes an access port for connection of the adapter with the catheter.

23. The trocar assembly of claim 22, further comprising a catheter connector configured to connect to the adapter.

24. The trocar assembly of claim 23, wherein the catheter connector comprises a male connecting portion and the adapter comprises a receiving portion to receive said male connecting portion.

25. The trocar assembly of claim 24, wherein the catheter connector includes a female connecting portion at an opposite end to the male connecting portion, the female connecting portion configured to receive an end of the catheter.

26. The trocar assembly of claim 25, wherein the catheter connector includes two male connecting portions and two female connecting portions.

27. The trocar assembly of claim 26, wherein the adapter includes two receiving portions to receive the two male connecting portions of the connector.

28. The trocar assembly of claim 22, wherein the handle comprises a pair of shells, mateable with each another in the housed configuration.

29. The trocar assembly of claim 28, wherein the handle comprises a snap-fit connection system for fastening the shells together in the housed configuration.

30. The trocar assembly of claim 28, wherein one of the shells comprises one or more projections receivable in corresponding recesses in the other shell portion to prevent relative movement between the shell portions in the housed configuration.

31. The trocar assembly of claim 30, wherein the handle comprises a release mechanism to release the one or more projections from their corresponding recesses.

32. A method for positioning a catheter in a target tissue site to deliver a medicament to a patient after haemorrhoid surgery, the method including:
providing a first catheter which extends from a proximal end to a distal end, the first catheter having a sidewall which defines an internal lumen, the distal end of the catheter having one or more apertures for the release of the medicament to the target tissue site;
making a first incision in the skin on one side of the anus;
making a lateral incision in the skin of the thigh of the patient;
connecting the proximal end of the first catheter to an end of a first trocar;
tunnelling the first trocar from the first incision through the tissue and through the lateral incision;
pulling the first trocar from the lateral incision until a desired length of the first catheter is pulled through the lateral incision, such that the proximal end of the first catheter extends from the lateral incision and the distal end of the first catheter extends from the first incision;
disconnecting the proximal end of the first catheter from the trocar;
connecting the distal end of the first catheter to a stimulator trocar, the stimulator trocar comprising an elongate body and a nerve stimulating electrode configured to stimulate nerves in the target tissue site;
advancing the stimulator trocar, together with the distal end of the first catheter, through the tissue of the patient;
actuating the nerve stimulator at a determined frequency;
adjusting the positioning of the stimulator trocar and the distal end of the first catheter until a physical contraction of the external anal sphincter is observed at a frequency that correlates with the determined frequency of the nerve stimulator;
identifying the location of the nerve stimulator and the distal end of the catheter where the physical contraction of the external anal sphincter is achieved as the first target tissue site;
disconnecting the stimulator trocar from the first catheter and withdrawing the stimulator trocar through the first incision leaving the distal end of the catheter implanted in the first target tissue site;
connecting the proximal end of the catheter to a reservoir of medicament and infusing the medicament through the internal lumen of the catheter to deliver the medicament to the first target tissue site.

33. The method of claim 32, wherein a second catheter is inserted into a second target tissue site, the second catheter extending from a proximal end to a distal end, the second catheter having a sidewall which defines an internal lumen, the distal end of the second catheter having one or more apertures for the release of the medicament to the second target tissue site.

34. A method for positioning first and second catheters in respective first and second target tissue sites to deliver a medicament to a patient after haemorrhoid surgery, the method including:
providing a first catheter which extends from a proximal end to a distal end, the first catheter having a sidewall which defines an internal lumen, the distal end of the first catheter having one or more apertures for the release of the medicament to the first target tissue site;
providing a second catheter which extends from a proximal end to a distal end, the second catheter having a sidewall which defines an internal lumen, the distal end of the second catheter having one or more apertures for the release of the medicament to the second target tissue site;
making a first incision in the skin on one side of the anus;
making a second incision in the skin on an opposite side of the anus to the first incision;
connecting the proximal end of the first catheter to a first trocar;
tunnelling the first trocar from the first incision through the tissue across the midline of the patient and through the second incision;
pulling the first trocar from the second incision until a desired length of the first catheter is pulled through the second incision, such that the proximal end of the first catheter extends from the second incision and the distal end of the first catheter extends from the first incision;
disconnecting the proximal end of the first catheter from the first trocar;
connecting the proximal end of the first catheter to a second trocar;
connecting the proximal end of the second catheter to the second trocar;
making a lateral incision in the skin of the thigh of the patient, the lateral incision being on the same side of the anus as the second incision;
tunnelling the second trocar from the second incision through the tissue and through the lateral incision;
pulling the second trocar from the lateral incision until a desired length of the first and second catheters is pulled through the lateral incision, such that the proximal ends of the first and second catheters extend from the lateral incision, the distal end of the first catheter extends from the first incision and the distal end of the second catheter extends from the second incision,
connecting the distal end of the first catheter to a stimulator trocar, the stimulator trocar comprising an elongate body and a nerve stimulator configured to stimulate nerves in the target tissue site;
advancing the stimulator trocar and the distal end of the first catheter through the tissue of the patient;
actuating the nerve stimulator at a determined frequency;
adjusting the positioning of the stimulator trocar and the distal end of the first catheter until a physical contraction of the external anal sphincter is observed at a frequency that correlates with the determined frequency;
identifying the location of the nerve stimulator and the distal end of the first catheter where physical contraction of the external anal sphincter is achieved as the first target tissue site;
disconnecting the first catheter from the stimulator trocar and withdrawing the stimulator trocar through the first incision leaving the distal end of the first catheter implanted in the first target tissue site;
connecting the distal end of the second catheter to the stimulator trocar;
advancing the stimulator trocar and the distal end of the second catheter through the second incision through tissue of the patient;
actuating the nerve stimulator at the determined frequency;
adjusting the positioning of the stimulator trocar and the distal end of the second catheter until a physical contraction of the external anal sphincter is observed at a frequency that correlates with the determined frequency;
identifying the location of the nerve stimulator and the distal end of the second catheter where physical contraction of the external anal sphincter is achieved as the second target tissue site;
disconnecting the second catheter from the stimulator trocar and withdrawing the stimulator trocar through the second incision leaving the distal end of the first catheter implanted in the second target tissue site;
connecting the proximal ends of the first and second catheters to a reservoir of medicament and infusing the medicament through the internal lumens of the first and second catheters to deliver the medicament to the first and second target tissue sites.

35. A surgical kit comprising:
first and second catheters configured for implantation in respective first and second target tissue sites of a patient, each catheter extending from a proximal end to a distal end and each having a sidewall which defines an internal lumen, said distal ends having one or more apertures;
a first tunnelling trocar configured for attachment to the distal end of the first catheter;
a second tunnelling trocar configured for simultaneous attachment to the distal ends of both the first and second catheters;
a stimulator trocar comprising an elongate body and a nerve stimulator configured to stimulate nerves in a target tissue site.

36. The surgical kit of claim 35 comprising two stimulator trocars.

37. The surgical kit of claim 35, further comprising a pump and a medicament reservoir.

38. The surgical kit of claim 35, further comprising:
a sheet for application to the patient's skin around the surgical site, the sheet having an adhesive surface for adhering to the skin of the patient and a series of frangible regions for removal of portions of the sheet.
